(19) United States
(12) Patent Application Publication
(10) Pub. No.: US 2012/0046600 A1
(43) Pub. Date: Feb. 23, 2012

(54) HIGH-SPEED ROTATIONAL ATHERECTOMY SYSTEM, DEVICE AND METHOD FOR LOCALIZED APPLICATION OF THERAPEUTIC AGENTS TO A BIOLOGICAL CONDUIT

(75) Inventors: Robert E. Kohler, Lake Elmo, MN (US); Brian Doughty, Edina, MN (US); Jody Lee Rivers, Elk River, MN (US)

(73) Assignee: CARDIOVASCULAR SYSTEMS, INC., St. Paul, MN (US)

(21) Appl. No.: 13/033,766

(22) Filed: Feb. 24, 2011

Related U.S. Application Data
(60) Provisional application No. 61/308,122, filed on Feb. 25, 2010.

(51) Int. Cl. A61M 37/00; A61B 17/3207

(52) U.S. Cl. 604/22

(57) ABSTRACT

The invention provides a system, device and method for localized application of therapeutic agents within a biological conduit. A preferred biological conduit comprises a blood vessel. A preferred device comprises a high-speed rotational atherectomy device having, in various embodiments, a flexible, elongate non-rotatable therapeutic agent delivery sheath having a lumen therethrough and a flexible, elongated, rotatable, drive shaft with at least one flexible eccentric enlarged abrading head disposed within lumen of the delivery sheath. The operator may actuate a controlled amount or dose of one or more therapeutic agents to release from the distal end of the delivery sheath lumen during high-speed rotation of the drive shaft. The therapeutic agent(s) is thus released into a turbulent fluidic environment resulting from high-speed rotation and orbital motion of the eccentric abrading head, which aids to drivingly urge the therapeutic agent(s) radially through the boundary layer of fluid flow in the conduit and into the target region of the conduit wall.

### BACKGROUND OF THE INVENTION

1. Field of the Invention

The invention relates to systems, devices and methods for treating biological conduits, e.g., blood vessels, with localized delivery of therapeutic agents.

2. Description of the Related Art

A variety of techniques and instruments have been developed for use in the removal or repair of tissue in biological conduits, e.g., without limitation, blood vessels and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes, leg pain and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patency of the opened artery. For non-calcified lesions, balloon angioplasty most often is used alone to open the artery, and stents often are placed to maintain patency of the opened artery. Studies have shown, however, that a significant percentage of patients who have undergone balloon angioplasty and had a stent placed in an artery experience stent restenosis—i.e., blockage of the stent which most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patency of the artery.

Several kinds of rotational atherectomy devices have been developed for attempting to remove stenotic material. In one type of device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. When rotated at high speeds, the abrasive segment is capable of removing stenotic tissue from an artery. Though this atherectomy device possesses certain advantages over the Auth device due to its flexibility, it also is capable only of opening an artery to a diameter about equal to the diameter of the enlarged abrading surface of the drive shaft since the device is not eccentric in nature.

U.S. Pat. No. 6,494,890 (Shturman) discloses an atherectomy device having a drive shaft with an enlarged eccentric section, wherein at least a segment of this enlarged section is covered with an abrasive material. When rotated at high speeds, the abrasive segment is capable of removing stenotic tissue from an artery. The device is capable of opening an artery to a diameter that is larger than the resting diameter of the enlarged eccentric section due, in part, to the orbital rotational motion during high speed operation. Since the enlarged eccentric section comprises drive shaft wires that are not bound together, the enlarged eccentric section of the drive shaft may flex during placement within the stenosis or during high speed operation. This flexion allows for a larger diameter opening during high speed operation, but may also provide less control than desired over the diameter of the artery actually abraded. In addition, some stenotic tissue may block the passageway so completely that the Shturman device cannot be placed therethrough. Since Shturman requires that the enlarged eccentric section of the drive shaft be placed within the stenotic tissue to achieve abrasion, it will be less effective in cases where the enlarged eccentric section is prevented from moving into the stenosis. The disclosure of U.S. Pat. No. 6,494,890 is hereby incorporated by reference in its entirety.

U.S. Pat No. 5,681,336 (Clement) provides an eccentric tissue removing burr with a coating of abrasive particles secured to a portion of its outer surface by a suitable binding material. This construction is limited, however because, as Clement explains at Col. 3, lines 53-55, that the asymmetrical burr is rotated at “lower speeds than are used with high speed ablation devices, to compensate for heat or imbalance.” That is, given both the size and mass of the solid burr, it is infeasible to rotate the burr at the high speeds used during atherectomy procedures, i.e., 20,000-200,000 rpm. Essentially, the center of mass offset from the rotational axis of the drive shaft would result in development of significant centrifugal force, exerting too much pressure on the wall of the artery and creating too much heat and excessively large particles.

Another method of treatment of occluded vessels may include the use of stents. Stents may be placed at the site of a stenosis and expanded to widen the vessel, remaining in position as a vessel implant.

No matter the technique used to open an occluded conduit, e.g., blood vessel, and restore normal fluid flow therethrough, one problem remains: restenosis. A certain percentage of the treated conduits and vessels will reocclude (restenose) after a period of time; occurring in as many as 40-50% of the cases. When restenosis does occur, the original procedure may be repeated or an alternative method may be used to reestablish fluid, e.g., blood, flow.

The relevant commonality shared by each of the above treatment methods is that each one results in some trauma to the conduit wall. Restenosis occurs for a variety of reasons; each involving trauma. Small clots may form on the arterial wall. Small tears in the wall expose the blood to foreign material and proteins which are highly thrombogenic. Resulting clots may grow gradually and may even contain growth hormones released by platelets within the clot. Moreover, growth hormones released by other cells, e.g., macrophages, may cause smooth muscle cells and fibroblasts in the affected region to multiply in an abnormal fashion. There may be an injury in the conduit wall due to the above methods that results in inflammation which may result in the growth of new tissue.

It is known that certain therapeutic substances may have a positive effect on prevention and/or inhibition of restenosis. Several difficulties present themselves in the application of these substances to the affected region in a therapeutic dose. For example, the region in need of treatment is very small and localized. Fluid, e.g., blood, flow in the conduit is continuous, resulting in a flow boundary along the wall which must be disrupted so that the therapeutic substances may reach the localized region of interest within a dose range considered therapeutic. The art fails to adequately provide a mechanism for breaking through this flow boundary to target the region of interest; electing instead generally to place the therapeutic substance into the general flow of the conduit, either by intravenous means or intra-lumen infusion, at a dose that is much higher than therapeutic since the majority of the therapeutic substance will simply flow downstream and either be absorbed systemically or eliminated as waste. For example, intravenous medications are delivered systemically by vein or orally, or regionally, e.g., through intra-lumen infusion without targeting the subject region. Such unnecessary systemic exposure results with unknown and unnecessary adverse results in regions, tissue, and/or organs that are distant from the region of interest. Clearly, systemic delivery and exposure is not well suited to treatment of diseases or conditions having a single intra-lumen region of interest.

The potential utility of localized application of a therapeutic dose of therapeutic agents is not limited to treatment of coronary arteries. Beyond coronary artery delivery, other sites of atherosclerosis, e.g., renal, iliac, femoral, distal leg and carotid arteries, as well as saphenous vein grafts, synthetic grafts and arterio-venous shunts used for hemodialysis would be appropriate biological conduits for a localized therapeutic substance delivery method and mechanism. Nor is the potential utility limited to blood vessels; any biological conduit having a region of interest amenable to treatment may benefit from such a treatment method and mechanism. The present invention may be used in any biological conduit where a catheter can be inserted. Such biological conduit includes, inter alia, blood vessels, urinary tract, coronary vasculature, esophagus, trachea, colon, and biliary tract.

The present invention overcomes these deficiencies.

### BRIEF SUMMARY OF THE INVENTION

The invention provides a system, device and method for localized application of therapeutic agents within a biological conduit. A preferred biological conduit comprises a blood vessel. A preferred device comprises a high-speed rotational atherectomy device having, in various embodiments, a flexible, elongate non-rotatable therapeutic agent delivery sheath having a lumen therethrough and a flexible, elongated, rotatable, drive shaft with at least one flexible eccentric enlarged abrading head disposed within lumen of the delivery sheath. The operator may actuate a controlled amount or dose of one or more therapeutic agents to release from the distal end of the delivery sheath lumen during high-speed rotation of the drive shaft. The therapeutic agent(s) is thus released into a turbulent fluidic environment resulting from high-speed rotation and orbital motion of the eccentric abrading head, which aids to drivingly urge the therapeutic agent(s) radially through the boundary layer of fluid flow in the conduit and into the target region of the conduit wall.

In this manner, application of a therapeutic dose of the therapeutic substance(s) at the affected region is achieved, while minimizing unwanted systemic exposure and the accompanying undesirable side effects. As a consequence, the need to administer super-therapeutic doses is eliminated.

An object of the invention is to provide a high-speed rotational atherectomy system, method and device for delivering a therapeutic dose of at least one therapeutic substance to an affected region on a biological conduit wall.

The figures and the detailed description which follow more particularly exemplify these and other embodiments of the invention.

### BRIEF DESCRIPTION OF THE DRAWINGS

The invention may be more completely understood in consideration of the following detailed description of various embodiments of the invention in connection with the accompanying drawings, which are as follows.

FIG. 1A is a velocity profile diagram showing a typical steady state Poiseuillean flow driven by constant pressure gradient.

FIG. 1B is a velocity profile diagram showing blood flow velocity within an exemplary biological conduit, an artery, averaged over the cardiac pulse.

FIG. 2 is a perspective view of one embodiment of one embodiment of the present invention;

FIG. 3A is a perspective view of one embodiment of an eccentric abrading head of the present invention;

FIG. 3B is a bottom view of one embodiment of an eccentric abrading head of the present invention;

FIG. 3C is a side cutaway view of one embodiment of an eccentric abrading head of the present invention;

FIG. 4 is a transverse cross-sectional view illustrating three different positions of the rapidly rotating eccentric abrading head of the rotational atherectomy device of the present invention;

FIG. 5 is a schematic diagram illustrating an exemplary spiral orbital path taken by an eccentric abrading head of the present invention as it removes stenotic tissue from an artery;

FIG. 6 is a graph illustrating the maximum centrifugal force generated by an eccentric abrading head of the present invention at various speeds of rotation;

FIG. 7 is a side partial cutaway view of one embodiment of the present invention;

FIG. 8 is an end cross sectional view of the embodiment of the present invention of FIG. 7;

FIG. 9 is a side partial cutaway view of one embodiment of the present invention;

FIG. 10 is an end cross sectional view of the embodiment of the present invention of FIG. 9; and

FIG. 11 is a side partial cutaway view of one embodiment of the present invention.

### DETAILED DESCRIPTION OF THE INVENTION, INCLUDING THE BEST MODE

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

For the purposes of the present invention, the following terms and definitions apply:

“Bodily disorder” refers to any condition that adversely affects the function of the body.

The term “treatment” includes prevention, reduction, delay, stabilization, and/or elimination of a bodily disorder, e.g., a vascular disorder. In certain embodiments, treatment comprises repairing damage cause by the bodily, e.g., vascular, disorder and/or intervention of same, including but not limited to mechanical intervention.

A “therapeutic agent” comprises any substance capable of exerting an effect including, but not limited to therapeutic, prophylactic or diagnostic. Thus, therapeutic agents may comprise anti-inflammatories, anti-infectives, analgesics, anti-proliferatives, and the like including but not limited to antirestenosis drugs. Therapeutic agent further comprises mammalian stem cells. Therapeutic agent as used herein further includes other drugs, genetic materials and biological materials. The genetic materials mean DNA or RNA, including, without limitation, of DNA/RNA encoding a useful protein, intended to be inserted into a human body including viral vectors and non-viral vectors. Viral vectors include adenoviruses, gutted adenoviruses, adeno-associated virus, retroviruses, alpha virus, lentiviruses, herpes simplex virus, ex vivo modified cells (e.g., stem cells, fibroblasts, myoblasts, satellite cells, pericytes, cardiomyocytes, skeletal myocytes, macrophage), replication competent viruses, and hybrid vectors. Non-viral vectors include artificial chromosomes and mini-chromosomes, plasmid DNA vectors, cationic polymers, graft copolymers, neutral polymers PVP, SP1017, lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor .alpha. and .beta., platelet derived endothelial growth factor, platelet derived growth factor, tumor necrosis factor .alpha., hepatocyte growth factor and insulin like growth factor), transcription factors, proteinkinases, CD inhibitors, thymidine kinase, and bone morphogenic proteins. These dimeric proteins can be provided as homodimers, heterodimers, or combinations thereof, alone or together with other molecules.

Therapeutic agents further includes cells that can be of human origin (autologous or allogeneic) or from an animal source (xenogeneic), genetically engineered, if desired, to deliver proteins of interest at the transplant site. Cells within the definition of therapeutic agents herein further include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Therapeutic agent also includes non-genetic substances, such as: anti-thrombogenic agents such as heparin, heparin derivatives, and urokinase; anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as heparin, antithrombin compounds, platelet receptor antagonists, anti-thrombin anticodies, anti-platelet receptor antibodies, aspirin, dipyridamole, protamine, hirudin, prostaglandin inhibitors, platelet inhibitors and tick antiplatelet peptides; vascular cell growth promotors such as growth factors, Vascular Endothelial Growth Factors, growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme, inhibitors including captopril and enalopril. The biologically active material can be used with (a) biologically non-active material(s) including a solvent, a carrier or an excipient, such as sucrose acetate isobutyrate, ethanol, n-methyl pymolidone, dimethyl sulfoxide, benzyl benxoate and benzyl acetate.

Further, “therapeutic agent” includes, in particular in a preferred therapeutic method of the present invention comprising the administration of at least one therapeutic agent to a procedurally traumatized, e.g., by an angioplasty or atherectomy procedure, mammalian vessel to inhibit restenosis. Preferably, the therapeutic agent is a cytoskeletal inhibitor or a smooth muscle inhibitor, including, for example, taxol and functional analogs, equivalents or derivatives thereof such as taxotere, paclitaxel, abraxane TM, coroxane TM or a cytochalasin, such as cytochalasin B, cytochalasin C, cytochalasin A, cytochalasin D, or analogs or derivatives thereof.

Additional specific examples of “therapeutic agents” that may be applied to a bodily lumen using various embodiments of the present invention comprise, without limitation:
L-Arginine;
Adipose Cells;
Genetically altered cells, e.g., seeding of autologous endothelial cells transfected with the beta-galactosidase gene upon an injured arterial surface;
Erythromycin;
Penicillin:
Heparin;
Aspirin;
Hydrocortisone;
Dexamethasone;
Forskolin;
GP IIb-IIIa inhibitors;
Cyclohexane;
Rho Kinsase Inhibitors;
Rapamycin;
Histamine;
Nitroglycerin;
Vitamin E;
Vitamin C;
Stem Cells;
Growth Hormones;
Hirudin;
Hirulog;
Argatroban;
Vapirprost;
Prostacyclin;
Dextran;
Erythropoietin;
Endothelial Growth Factor;
Epidermal Growth Factor;
Core Binding Factor A;
Vascular Endothelial Growth Factor;
Fibroblast Growth Factors;
Thrombin;
Thrombin inhibitor; and
Glucosamine, among many other therapeutic substances.

The device of the present invention can be used to apply the biologically active material to any surface of a biological conduit where a catheter can be inserted. Such biological conduit includes, inter alia, blood vessels, urinary tract, coronary vasculature, esophagus, trachea, colon, and biliary tract.

One particular problem with known local delivery mechanisms and methods that do not make use of expanding delivery members comprising, e.g., barbs, needles and the like to mechanically delivery therapeutic agents to the wall of a biological conduit is enabling the agents to move from the point of release radially outwardly to the conduit wall. This is because the flow within the conduit may generally turbulent or laminar, depending on the type of conduit under consideration, but in both cases if the turbulent or laminar region can be successfully navigated, a boundary layer adjacent the conduit wall exists comprising forces which must be broken through in order to reach the conduit wall.

Consider, as a non-limiting general case, arterial blood flow which is completely bound by a solid surface, i.e. the arterial wall, and is called an internal flow. Internal flows may be characterized as laminar or turbulent. In the laminar case, flow structure is characterized by smooth motion in layers. Laminar flow has no turbulent kinetic energy. Flow structure in the turbulent case is characterized by random, three-dimensional motions of fluid particles superimposed on the mean motion.

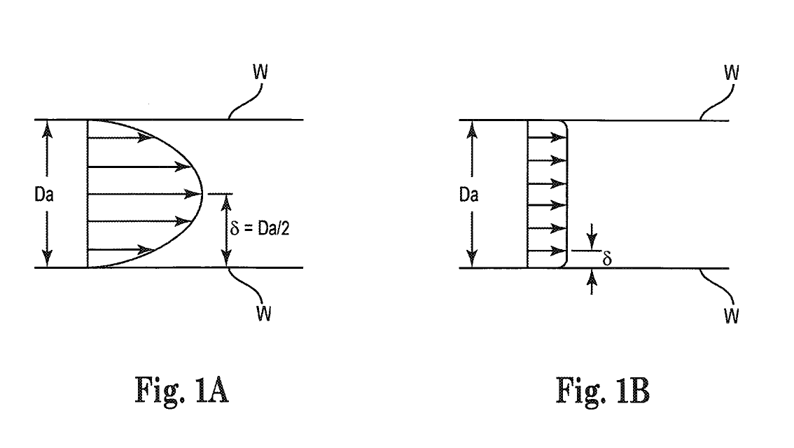

The most basic fluid mechanic equations predict the behavior of internal pipe flows under a uniform and constant pressure. Under these conditions the flow is Poiseuillean. FIG. 1A is a velocity profile diagram showing a typical steady state Poiseuillean flow driven by constant pressure. The velocity of the fluid across the pipe is shown in FIG. 1A by the parabolic curve and corresponding velocity vectors. The velocity of the fluid in contact with the wall of the pipe is zero. The boundary layer is the region of the flow in contact with the pipe surface in which viscous stresses are dominant. In the steady state Poiseuillean flow, the boundary layer develops until it reaches the pipe center line. For example, the boundary layer thickness, δ, in FIG. 1A is one half of the diameter of the pipe, Dₐ. FIG. 1A is introduced for comparison purposes to show the difference between standard Poiseuillean flow and the flow which develops within an artery.

Under conditions of Poiseuillean flow, the Reynolds number, Re, can be used to characterize the level of turbulent kinetic energy. The Reynolds number, Re, is the ratio of inertial forces to viscous forces as is well known in the art. For Poiseuillean flows, Reynolds numbers, Re, must be greater than about 2300 to cause a laminar to turbulent transition. Further, under conditions of high Reynolds numbers (>2000), the boundary layer is receptive to “tripping”. Tripping is a process by which a small perturbation in the boundary layer amplifies to turbulent conditions. The receptivity of a boundary layer to “tripping” is proportional to the Reynolds, Re, number and is nearly zero for Reynolds, Re, numbers less than 2000.

However, the blood flow in the arteries is induced by the beating heart and is pulsatile, complicating the turbulent fluid mechanics analysis above. Although very high velocities are reached at the peak of the pulse, the high velocity occurs for only a small portion of the cycle. In fact, the velocity of the blood reaches zero in the carotid artery at the end of a pulse and temporarily reverses.

Because of the relatively short duration of the cardiac pulse, the blood flow in the arteries does not develop into classic Poiseuillean flow. FIG. 1B is a velocity profile diagram showing blood flow velocity within an artery averaged over the cardiac pulse. Notice that the majority of the flow within the artery has the same velocity. The character of the pulsed flow in an artery of diameter, Dₐ, is determined by the value of a dimensionless parameter called the Womersley number. The Womersley number expresses the ratio between oscillatory inertia forces and viscous shear forces and is also proportional to the interior diameter of the artery and inversely proportional to the thickness of the boundary layer as the skilled artisan will readily understand.

The Womersley number is known to be relatively high (N_{w}=15-20) in the aorta and in the common carotid artery (N_{w}=6-10). The relatively high Womersley numbers results in the relatively blunt velocity profile in contrast to the parabolic profile of the steady state viscous Poiseuillean flow. In other words, the arterial flow is predominately composed of an inviscid “free stream” and a very thin viscous boundary layer adjacent to the artery wall. “Free stream” refers to the flow that is not affected by the presence of the solid boundaries and in which the average velocity remains fairly constant as a function of position within the artery. The motion in the boundary layer is mainly the result of the balance between inertia and viscous forces, while in the free stream, the motion is the result of the balance between inertia and pressure forces. In FIG. 1B, notice that the boundary layer where the flow velocity decays from the free stream value to zero is very thin, typically ⅙ to 1/20 of the diameter of the artery, as opposed to one half of the diameter of the artery in the Poiseuillean flow condition, though the forces therein are relatively significant and must be overcome to reach the conduit wall W.

Thus, a therapeutic agent released within the free stream must overcome the directional laminar flow to move toward the conduit wall W, generally 90 degrees away from the directional laminar flow. Once successfully through the free stream laminar flow region, the therapeutic agent then must overcome the boundary layer motion and turbulence therein, in order to ultimately reach the conduit wall W.

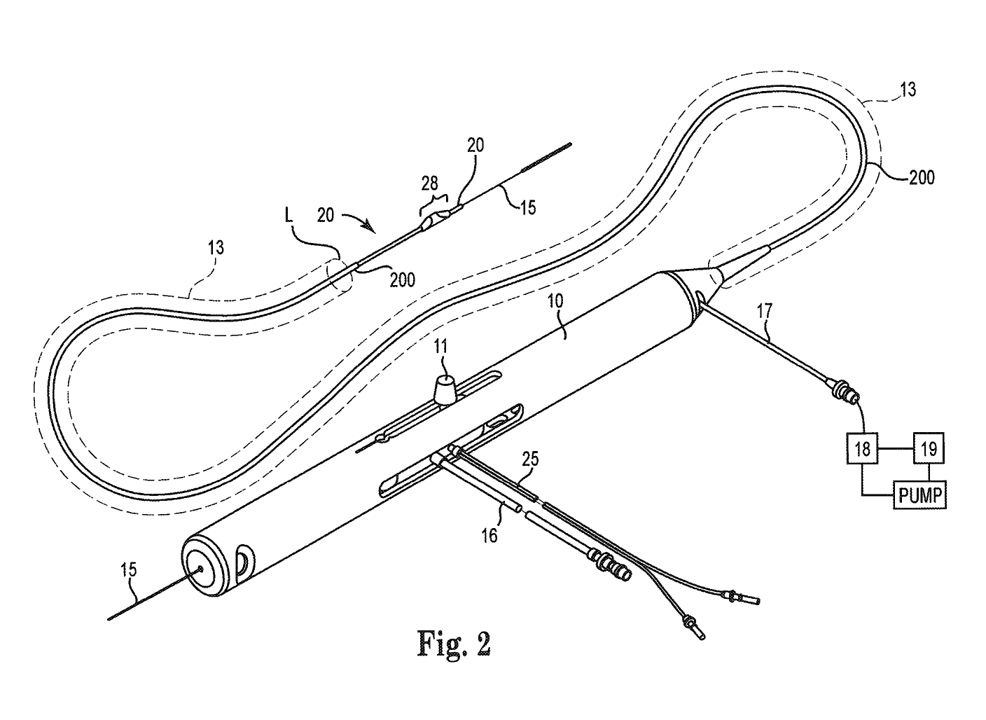

FIG. 2 illustrates one embodiment of a rotational atherectomy device according to the present invention. The device includes a handle portion 10, an elongated, flexible drive shaft 20 having an eccentric abrading head 28, an elongated, flexible therapeutic agent delivery sheath 200 having a lumen therethrough, and an elongated catheter 13, illustrated with dashed lines, extending distally from the handle portion 10. The drive shaft 20 is constructed from helically coiled wire as is known in the art and the abrading head 28 is fixedly attached thereto. The catheter 13 has a lumen L within which the therapeutic agent delivery sheath 200 is slidably disposed. Drive shaft 20 is rotatably and slidably disposed within the lumen of therapeutic agent delivery sheath 200.

In one embodiment, the therapeutic substance delivery sheath 200 may be slidably disposed within the catheter lumen L, allowing the operator to axially translate the distal opening of the therapeutic substance delivery sheath 200 to various points within the catheter lumen L or distally outside of the catheter lumen L. The inner diameter of lumen of therapeutic agent delivery sheath 200 is smaller than the outer diameter of the eccentric abrading head 28 in certain embodiments. Thus, delivery sheath 200 may not be, in these embodiments, slidably translated over the eccentric abrading head 28.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20 The handle 10 also desirably includes a control knob 11 for advancing and retracting the turbine and drive shaft 20 with respect to the catheter 13 and the body of the handle. A therapeutic substance reservoir 18 may be provided, either separately as in the form of a plungeable syringe, actuated by the operator, the syringe being in fluid communication with the lumen of therapeutic agent delivery sheath 200 as illustrated and described in commonly assigned application Ser. No. 13/029,477, entitled Systems and Methods for Mixing Therapeutic Agents Before and/or During Administration. The entire contents of application Ser. No. 13/029,477 are hereby incorporated by reference. Alternatively, therapeutic substance reservoir 18 may be coupled with a pump, as illustrated in FIG. 2 and reservoir 18 and pump may be operatively connected with a controller 19 for controlling actuation of pump. In either case, or any equivalent cases, reservoir 18 is in fluid communication with the lumen of delivery sheath 200.

Still more alternatively, low shearing methods, including but not limited to distal loading of the therapeutic agent(s) within delivery sheath 200, or other delivery device, may be desirable. Thus, the entire contents of commonly assigned application Ser. No. 13/026,567, entitled Device and Methods for Low Shearing Local Delivery of Therapeutic Agents to the Wall of a Bodily Lumen, is incorporated herein by reference.

Actuation of pump for introducing therapeutic substance(s) into the drive shaft lumen may be controlled by a separate controller knob located on the handle 10 or by a separate controller 19 mounted in operative communication with the pump and/or therapeutic substance reservoir 18. It will be readily apparent to the skilled artisan that the dosing of the therapeutic substance(s), advanced through the lumen of the therapeutic substance delivery sheath 200 from the therapeutic substance reservoir 18 and to a point proximal the abrading head 28 for release therefrom prior to high-speed rotation of the eccentric abrading head 28 and/or during high-speed rotation of the eccentric abrading head 28, may be monitored and controlled in many ways. For example, only a known dosage of therapeutic substance(s) may be added to the therapeutic substance reservoir 18 and/or a gauge may be employed to assist the operator in monitoring the amount of therapeutic substance moving through fluid supply line 17. All such known methods of monitoring the amount of fluid flow are within the scope of the present invention.

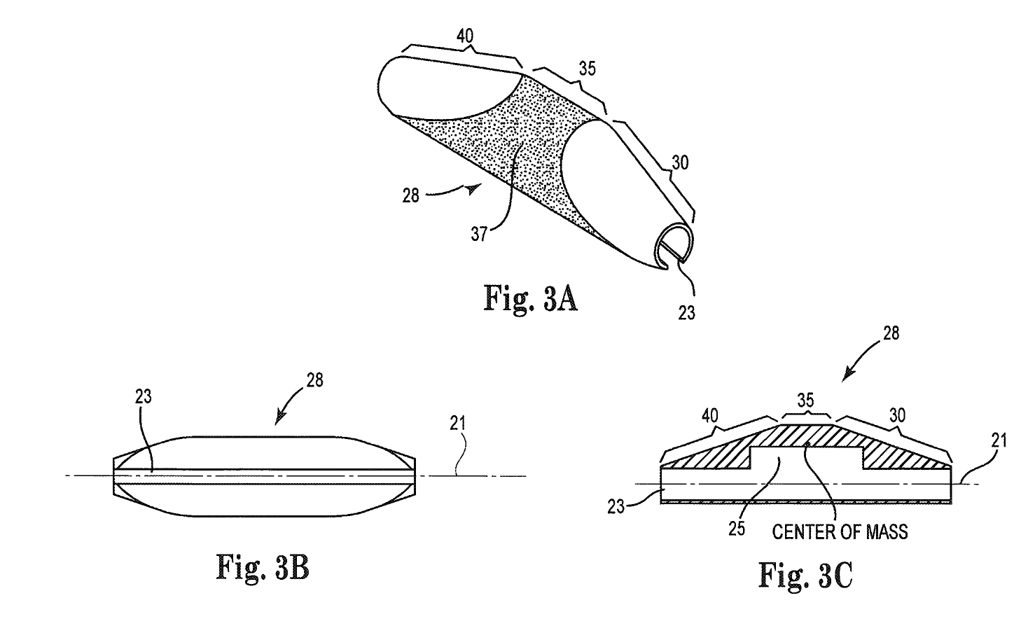

Turning now to FIGS. 3A, 3B and 3C, one embodiment of an eccentric enlarged abrading head 28 of the rotational atherectomy device of the invention will be discussed.

The drive shaft 20 has a rotational axis 21, which is coaxial with the guide wire 15, the guide wire 15 being disposed within the lumen 19 of the drive shaft 20 as illustrated in FIG. 1. Eccentric abrading head 28 is disposed on the drive shaft 20, near the distal end of the drive shaft 20.

The abrading head 28 may comprise at least one tissue removing surface 37 on the external surface(s) of the intermediate portion 35, the distal portion 40 and/or the proximal portion 30 to facilitate abrasion of the stenosis during high speed rotation. The tissue removing surface 37 may comprise a coating of an abrasive material 24 bound to the external surface(s) of the intermediate portion 35, the distal portion 40 and/or the proximal portion 30 of abrading head 28. The abrasive material may be any suitable material, such as diamond powder, fused silica, titanium nitride, tungsten carbide, aluminum oxide, boron carbide, or other ceramic materials. Preferably the abrasive material is comprised of diamond chips (or diamond dust particles) attached directly to the tissue removing surface(s) by a suitable binder 26—such attachment may be achieved using well known techniques, such as conventional electroplating or fusion technologies (see, e.g., U.S. Pat. No. 4,018,576). Alternately the external tissue removing surface may comprise mechanically or chemically roughening the external surface(s) of the intermediate portion 35, the distal portion 40 and/or the proximal portion 30 to provide a suitable abrasive tissue removing surface 37. In yet another variation, the external surface may be etched or cut (e.g., with a laser) to provide small but effective abrading surfaces. Other similar techniques may also be utilized to provide a suitable tissue removing surface 37.

An at least partially enclosed lumen or slot 23 may be provided longitudinally through the enlarged abrading head 28 along the rotational axis 21 of the drive shaft 20 for securing the abrading head 28 to the drive shaft 20 in a manner well known to those skilled in the art. In the embodiment shown, a hollowed section 25 is provided to lessen the mass of the abrading head 28 to facilitate atraumatic abrasion and improve predictability of control of the orbital pathway of the abrading head 28 during high speed, i.e., 20,000 to 200,000 rpm, operation. In this embodiment, the abrading head 28 may be fixedly attached to the drive shaft 20, wherein the drive shaft comprises one single unit. The size and shape of the hollowed section 25 may be modified to optimize the orbital rotational path of the abrading head 28 for particularly desirable rotational speeds. Those skilled in the art will readily recognize the various possible configurations, each of which is within the scope of the present invention.

The embodiment of FIGS. 3A-3C illustrates the proximal portion 30 and distal portion 40 of approximately symmetrical shape and length. Alternate embodiments may increase the length of either the proximal portion 30 or the distal portion 40, to create an asymmetrical profile.

The eccentric enlarged abrading head 28 has a center of mass that is spaced radially away from the longitudinal rotational axis 21 of the drive shaft 20. As will be described in greater detail below, offsetting the center of mass from the drive shaft's axis of rotation 21 provides the enlarged abrading head 28 with an eccentricity that permits it to open an artery to a diameter substantially larger, than the nominal diameter of the enlarged eccentric abrading head 28, preferably the opened diameter is at least twice as large as the nominal resting diameter of the enlarged eccentric abrading head 28. The magnitude of the offset of the center of mass from the rotational axis 21 of the drive shaft 20 may be manipulated by modifying, e.g., the hollow space 25 and/or the density of the materials used in manufacturing eccentric abrading head 28 and/or the geometry of the eccentric abrading head 28.

Additional variations of the eccentric enlarged abrading head 28 are also possible, including an arrangement whereby the wire turns of the drive shaft are enlarged on one side of the drive shaft but not the opposing side, creating an offset of the center of mass from the axis of rotation. This arrangement is disclosed within U.S. Pat. No. 6,494,890 to Shturman, the entire contents of which is hereby incorporated herein by reference. The significant part of the eccentric enlarged abrading head 28 of the present invention and its various embodiments is that eccentricity is created, i.e., that the center of mass of the eccentric enlarged abrading head is offset from the axis of rotation of the drive shaft. Such eccentricity drives an orbital pattern of rotation for the eccentric enlarged abrading head 28 as will be discussed further and which is a significant element of the various embodiments of the present invention.

Accordingly, it should be understood that, as used herein, the word “eccentric” is defined and used herein to refer to either a difference in location between the geometric center of the eccentric abrading head 28 and the rotational axis 21 of the drive shaft 20, or to a difference in location between the center of mass of the enlarged abrading head 28 and the rotational axis 21 of the drive shaft 20. Either such difference, at the proper rotational speeds, will enable the eccentric abrading head 28 to open a stenosis to a diameter substantially greater than the nominal, or resting, diameter of the eccentric abrading head 28. Moreover, for an eccentric abrading head 28 having a shape that is not a regular geometric shape, the concept of “geometric center” can be approximated by locating the mid-point of the longest chord which is drawn through the rotational axis 21 of the drive shaft 28 and connects two points on a perimeter of a transverse cross-section taken at a position where the perimeter of the eccentric abrading head 28 has its maximum length.

The abrading head 28 of the rotational atherectomy device of the invention may be constructed of stainless steel, tungsten, titanium or similar material. The abrading head 28 may be a single piece unitary construction or, alternatively, may be an assembly of two or more abrading head components fitted and fixed together to achieve the objects of the present invention.

The extent to which a stenosis in an artery can be opened to a diameter larger than the nominal diameter of the eccentric enlarged abrading head of the present invention depends on several parameters, including the shape of the eccentric enlarged abrading head, the mass of the eccentric enlarged abrading head, the distribution of that mass and, therefore, the location of the center of mass within the abrading head with respect to the rotational axis of the drive shaft, and the speed of rotation.

The speed of rotation is a significant factor in determining the centrifugal force with which the tissue removing surface of the enlarged abrading head is pressed against the stenotic tissue, thereby permitting the operator to control the rate of tissue removal. Control of the rotational speed also allows, to some extent, control over the maximum diameter to which the device will open a stenosis. Applicants have also found that the ability to reliably control the force with which the tissue removing surface is pressed against the stenotic tissue not only permits the operator to better control the rate of tissue removal but also provides better control of the size of the particles being removed.

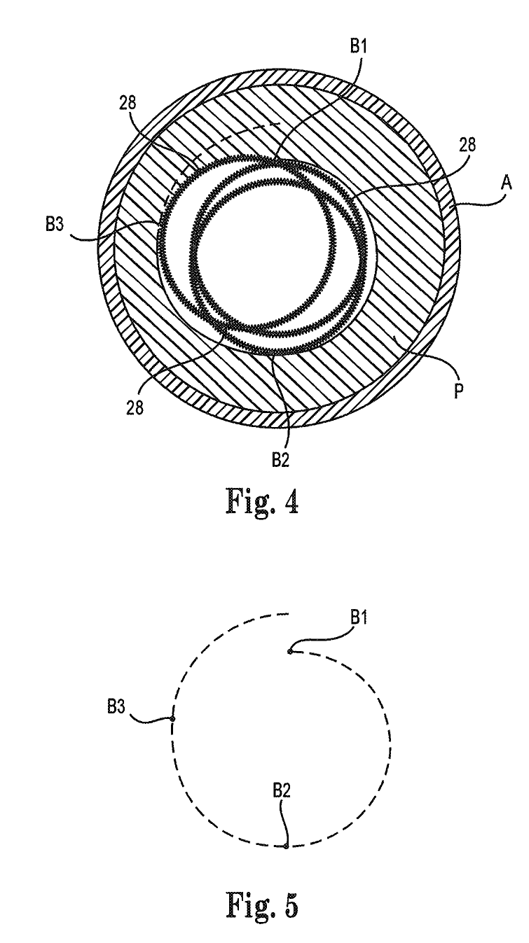

FIGS. 4 and 5 illustrate the generally spiral orbital path taken by various embodiments of the eccentric abrading head 28 of the present invention, the abrading head 28 shown relative to the guide wire 15 over which the abrading head 28 has been advanced. The pitch of the spiral path in FIGS. 4 and 5 is exaggerated for illustrative purposes—in reality, each spiral path of the eccentric abrading head 28 removes only a very thin layer of tissue via the abrading head 28, and many, many such spiral passes are made by the eccentric abrading head 28 as the device is repeatedly moved forward and backward across the stenosis to fully open the stenosis. FIGS. 4 and 5 show schematically three different rotational positions of the eccentric abrading head 28 of a rotational atherectomy device of the invention. At each position the abrasive surface of the eccentric enlarged abrading head 28 contacts the plaque “P” to be removed—the three positions are identified by three different points of contact with the plaque “P”, those points being designated in the drawing as points B1, B2, and B3. Notice that at each point it is generally the same portion of the abrasive surface of the eccentric abrading head 28 that contacts the tissue—the portion of the tissue removing surface 37 that is radially most distant from the rotational axis of the drive shaft.

Although not wishing to be constrained to any particular theory of operation, applicants believe that offsetting the center of mass from the axis of rotation produces an “orbital” movement of the enlarged abrading head, the diameter of the “orbit” being controllable by varying, inter alia, the rotational speed of the drive shaft. Applicants have empirically demonstrated that by varying the rotational speed of the drive shaft one can control the centrifugal force urging the tissue removing surface of the eccentric abrading head 28 against the surface of the stenosis. The centrifugal force can be determined according to the formula:

F_{c}=mΔx(πn/30)²

where F_{c} is the centrifugal force, m is the mass of the eccentric abrading head, Δx is the distance between the center of mass of the eccentric abrading head and the rotational axis of the drive shaft, and n is the rotational speed in revolutions per minute (rpm). Controlling this force F_{c} provides control over the rapidity with which tissue is removed, control over the maximum diameter to which the device will open a stenosis, and improved control over the particle size of the tissue being removed. Controlling force F_{c} also provides control over the impaction of therapeutic agent(s) within the influence of the high-speed rotational eccentric abrading head 28, as the agent(s) may be radially driven by the forces created during the orbital motion of the eccentric abrading head 28 into the biological conduit wall.

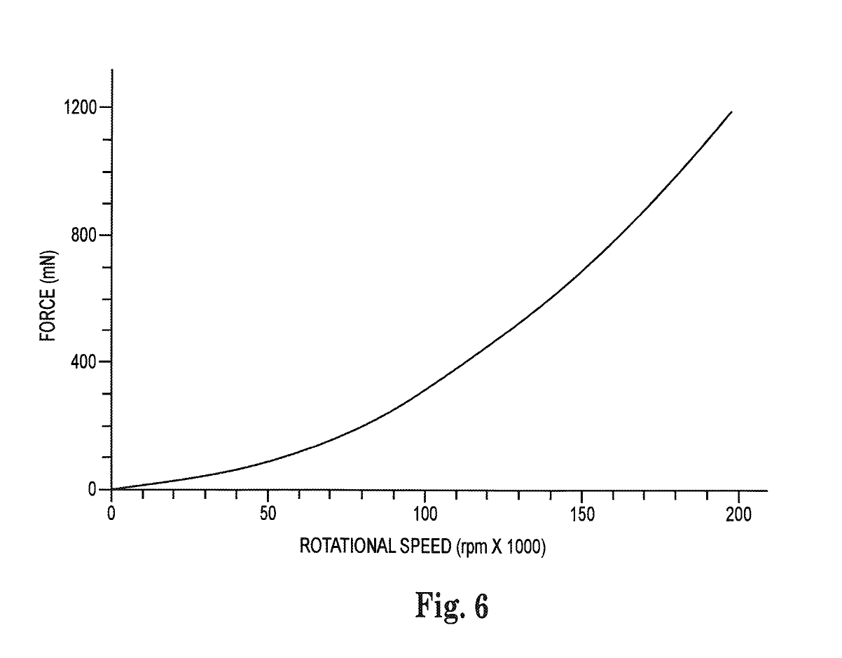

The graph shown in FIG. 6 illustrates calculations of the maximum centrifugal force F_{c} with which a tissue removing surface of an exemplary eccentric enlarged diameter section, having a maximum diameter of about 1.75 mm, can press against a surface of a stenosis at rotational speeds up to about 200,000 rpm. Controlling this force F_{c} provides control over the rapidity with which tissue is removed, control over the maximum diameter to which the device will open a stenosis, and improved control over the particle size of the tissue being removed. Utilizing this force F_{c} to assist in the delivery of therapeutic substances delivered into the orbital path of the high-speed rotational abrading head 28 is one focus of the present invention in its various embodiments.

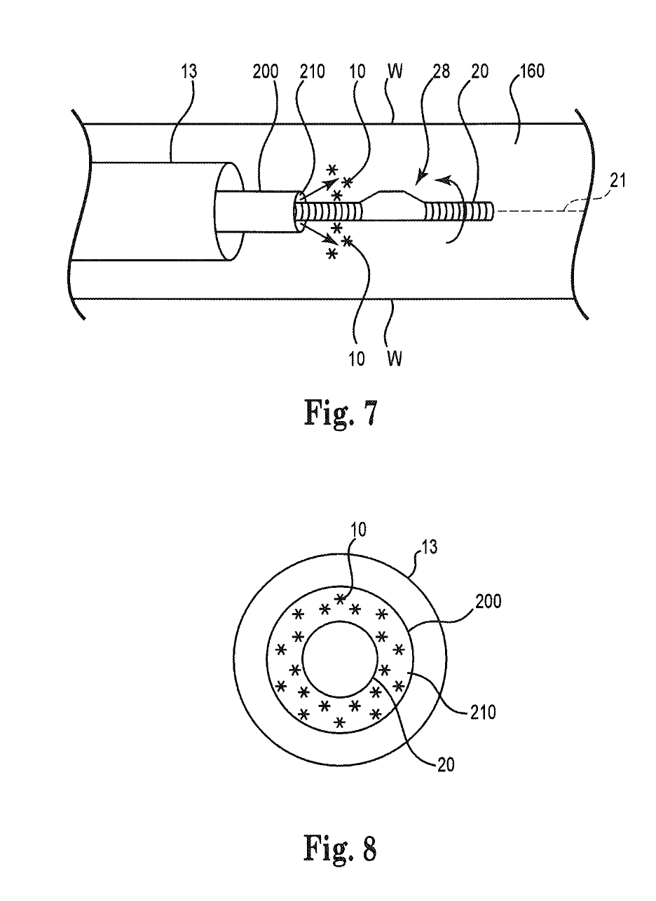

Turning now to FIGS. 7 and 8, the embodiment of the present invention illustrated in FIG. 2 is shown in closer detail. Catheter 13 is positioned within biological conduit 160. Therapeutic agent delivery sheath 200, having a lumen therethrough in fluid communication with therapeutic agent reservoir 18, is slidably positioned within the lumen of catheter 13, the distal end of delivery sheath 200 protruding distally from the lumen of catheter 13. Drive shaft 20 is rotatably positioned within lumen of delivery sheath 200, with the eccentric abrading head 28 disposed distal to the distal end of delivery sheath 200. A therapeutic agent delivery lumen 210 is defined by the space between the drive shaft and the therapeutic agent delivery sheath and is in fluid communication with therapeutic agent reservoir 18.

The at least one therapeutic agent 10 is illustrated as being released from the lumen of delivery sheath 200 while eccentric abrading head 28 is rotating at high speed, though such release may occur before initiation of the high-speed rotation of eccentric abrading head 28. The release of therapeutic agent(s) 10 may be achieved by actuating pump which, in turn, pumps the therapeutic agent(s) 10 from therapeutic reservoir 18 through therapeutic agent delivery lumen 210 to the distal end of the sheath 200 where the agent(s) 10 are released into the environment within the biological conduit 160. This actuation may be initiated either manually or automatically by controller 19.

In certain embodiments, the therapeutic agent(s) 10 may be transported within, and delivered from, the lumen defined as the space between catheter 13 and therapeutic agent delivery sheath 200, while the lumen within sheath 200 is utilized to deliver saline and/or lubricant through a separate input line as the skilled artisan will readily understand.

As discussed supra, the centrifugal forces generated by the high-speed orbital rotational motion of the eccentric abrading head 28 create radial forces. The therapeutic agent(s) 10 are released from the distal end of lumen of therapeutic agent delivery sheath 200 into this environment and are thereby urged radially outward and driven or impacted into the wall W of biological conduit 160. The radial forces generated by the high-speed rotational motion of abrading head 28 are sufficiently large to enable the therapeutic agent(s) 10 to move through the free stream laminar flow region or, alternatively though a turbulent flow region to reach the boundary layer adjacent the wall W of conduit 160 present during normal flow of the liquid, e.g., blood, within the conduit 160. These radial forces are further sufficient to enable the therapeutic agent(s) 10 to move radially through the boundary layer to impact the wall W where the agents' therapeutic potential is realized.

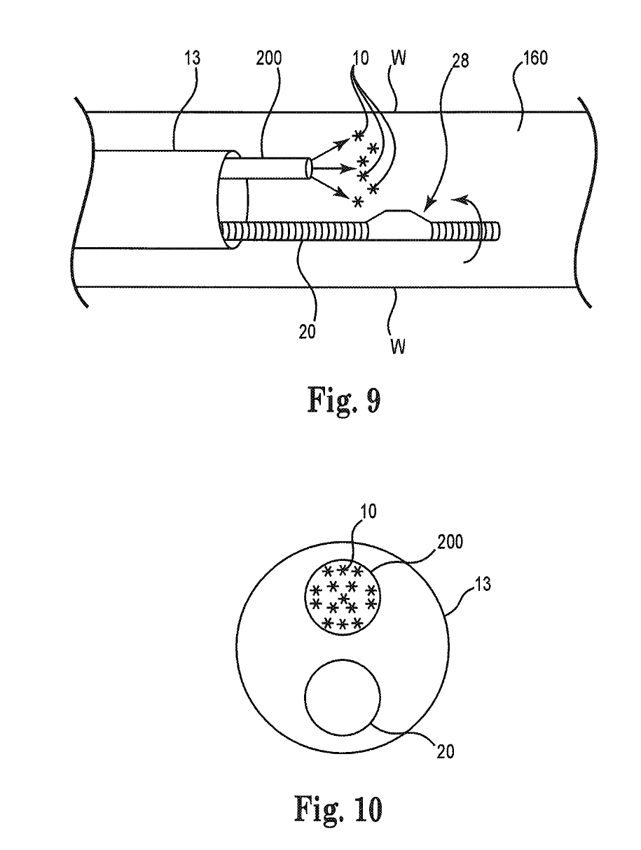

FIGS. 9 and 10 provide illustration of an alternate embodiment to the system of FIG. 2. Catheter 13 with lumen therethrough is positioned within biological conduit 160. Drive shaft 20 with eccentric abrading head 28 attached thereto is slidably and rotatably disposed within lumen of catheter 13. Therapeutic agent delivery sheath 200 is slidably disposed within lumen of catheter 13. As illustrated, the distal end of delivery sheath 200 is disposed proximal the eccentric abrading head 28 which is shown as rotating. The release of therapeutic agent(s) 10 may be achieved by actuating pump which, in turn, pumps the therapeutic agent(s) 10 from therapeutic reservoir 18 through lumen of therapeutic agent delivery sheath 200 to the distal end of the sheath 200 where the agent(s) 10 are released into the environment within the biological conduit 160. This actuation may be initiated either manually or automatically by controller 19.

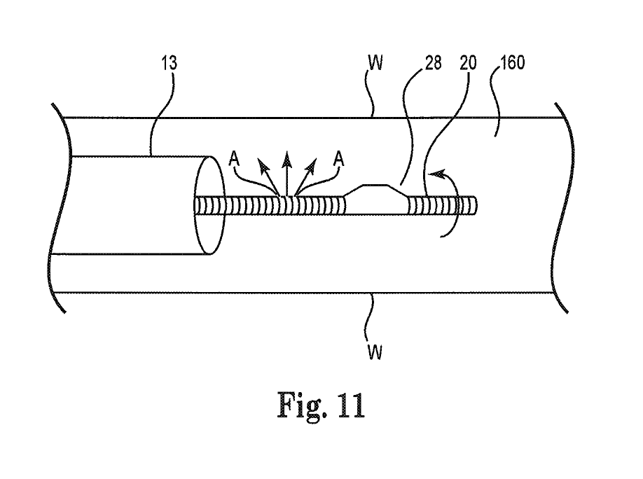

FIG. 11 illustrates another alternate embodiment of the present invention, wherein catheter 13 is positioned within the biological conduit 160 and drive shaft 20 is slidably and rotatably disposed within the lumen of catheter 13. In this embodiment, drive shaft 20 comprises a lumen which is in fluid communication with an external therapeutic agent reservoir, pump and controller such as that illustrated in FIG. 2. Drive shaft 20 further comprises at least one aperture A disposed near the eccentric abrading head 28, the at least one aperture in fluid communication with the lumen of drive shaft 20. As illustrated the at least one aperture A is located proximal to the eccentric abrading head 28, but such aperture A may be alternatively located distal to the eccentric abrading head 28. Still more alternatively, the at least one aperture A may be located both proximally and distally to the eccentric abrading head 28. Release of the therapeutic agent(s) 10 through the at least one aperture A may be achieved by actuating the pump, either manually or automatically by, e.g., a controller in operative communication with the pump, which in turn pumps the therapeutic agent(s) 10 from the therapeutic agent reservoir into the lumen of the drive shaft 20 and, ultimately, the agent(s) 10 is released from the at least one aperture A before and/or during high-speed orbital rotation of the eccentric abrading head 28.

In all embodiments, the illustrations portray the release of the at least one therapeutic agent (10) occurring during high-speed rotation of the drive shaft 20 and the eccentric abrading head 28, so that the agent(s) 10 are introduced directly into the radial forces created by the high-speed orbital rotational motion of eccentric abrading head 28. Each embodiment also, however, contemplates the release of the at least one therapeutic agent (10) at a point before the initiation of high-speed rotation of the drive shaft 20 and eccentric abrading head 28. Thus, the release of the at least one therapeutic agent (10) may occur in various embodiments of the present invention, before initiation of, and/or during, the high-speed rotation of the drive shaft 20 and the eccentric abrading head 28. In each of these cases, the centrifugal forces generated will urge the agent(s) 10 radially through the flowing liquid and boundary layer toward the conduit wall W.

Moreover, the agent(s) 10 may be subjected to a generally radially directed impact force if the agent(s) 10 contacts the high-speed rotational eccentric abrading head 28 and/or the drive shaft 20. This impact force will, in combination with the radial centrifugal forces created by the high-speed orbital rotational motion of the eccentric abrading head 28, drivingly urge the agent(s) 10 through the flowing liquid, e.g., blood, in the conduit 160 and into the wall W.

A method according to the present invention comprises: providing an elongate flexible therapeutic agent delivery sheath in fluid communication with a therapeutic agent reservoir and pump; providing an elongated, flexible rotatable drive shaft; providing an eccentric abrading head near the distal end of the drive shaft; providing a source of high-speed rotational power in operative connection with the drive shaft; inserting the therapeutic agent delivery sheath and drive shaft into the biological conduit near a region of interest; pumping the therapeutic agent through the lumen of the delivery sheath; releasing the therapeutic agent into the biological conduit near the eccentric abrading head; rotating the drive shaft and eccentric abrading head at high speed to drive the eccentric abrading head in an orbital path; creating centrifugal forces within the lumen around the eccentric abrading head; driving the therapeutic agent radially outward toward the biological conduit wall; and impacting the therapeutic agent in the biological conduit wall.

An alternative method may comprise rotating the drive shaft and eccentric abrading head before releasing the therapeutic agent into the biological conduit near the rotating eccentric abrading head. Still another alternative comprises impacting at least some of the released therapeutic agents with the orbitally rotating eccentric abrading head to drive the therapeutic agent radially outward toward the biological conduit wall and impacting the therapeutic agent in the biological conduit wall. Yet another alternate embodiment comprises exposing the released therapeutic agents, released either before and/or during the initiation of high-speed rotation of the eccentric abrading head, to a combination of impacting with the orbitally rotating eccentric abrading head and the centrifugal forces created by the rotating eccentric abrading head to drive the therapeutic agent radially outward toward the biological conduit wall and impact the therapeutic agent in the biological conduit wall.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A high-speed rotational atherectomy device for local delivery of at least one therapeutic agent to a biological conduit, comprising:
a guide wire having a maximum diameter less than the diameter of the artery;
a flexible elongated, rotatable drive shaft advanceable over the guide wire, the drive shaft having a rotational axis, the drive shaft rotatable at high rotational speeds;
an eccentric abrading head attached to the drive shaft, wherein the abrading head defines a drive shaft lumen therethrough and a hollow cavity, the drive shaft at least partially traversing the drive shaft lumen and wherein the at least one eccentric abrading head has a center of mass which is spaced radially away from the rotational axis of the drive shaft;
a flexible elongated catheter comprising a lumen,
a therapeutic agent delivery sheath comprising a lumen, the lumen comprising a distal end, the drive shaft slidably and rotatably disposed within the lumen of the therapeutic agent delivery sheath, and the therapeutic agent delivery sheath slidably disposed within the lumen of the catheter;
a therapeutic agent delivery lumen defined by the space between the drive shaft and the therapeutic agent delivery sheath; and
a therapeutic agent reservoir comprising the at least one therapeutic agent and in fluid communication with the therapeutic agent delivery lumen.

2. The device of claim 1, further comprising:
a pump in fluid communication with the therapeutic agent reservoir; and
a controller in operative communication with the pump and the therapeutic agent reservoir.

3. A high-speed rotational atherectomy device for local delivery of at least one therapeutic agent to a biological conduit, comprising:
a guide wire having a maximum diameter less than the diameter of the artery;
a flexible elongated, rotatable drive shaft advanceable over the guide wire, the drive shaft having a rotational axis, the drive shaft rotatable at high rotational speeds;
an eccentric abrading head attached to the drive shaft, wherein the abrading head defines a drive shaft lumen therethrough and a hollow cavity, the drive shaft at least partially traversing the drive shaft lumen and wherein the at least one eccentric abrading head has a center of mass which is spaced radially away from the rotational axis of the drive shaft;
a flexible elongated catheter comprising a lumen,
a therapeutic agent delivery sheath comprising a lumen, the lumen comprising a distal end, the drive shaft slidably and rotatably disposed within the lumen of the catheter, and the therapeutic agent delivery sheath slidably disposed within the lumen of the catheter; and
a therapeutic agent reservoir comprising at least one therapeutic agent and in fluid communication with the lumen of the therapeutic agent delivery sheath.

4. A high-speed rotational atherectomy device for local delivery of at least one therapeutic agent to a biological conduit, comprising:
a guide wire having a maximum diameter less than the diameter of the artery;
a flexible elongated, rotatable drive shaft advanceable over the guide wire, the drive shaft having a rotational axis, the drive shaft rotatable at high rotational speeds, the drive shaft further comprising a lumen therethrough and at least one aperture;
an eccentric abrading head attached to the drive shaft, wherein the abrading head defines a drive shaft lumen therethrough and a hollow cavity, the drive shaft at least partially traversing the drive shaft lumen and wherein the at least one eccentric abrading head has a center of mass which is spaced radially away from the rotational axis of the drive shaft, wherein the at least one aperture is disposed near the eccentric abrading head;
a flexible elongated catheter comprising a lumen, the drive shaft slidably and rotatably disposed within the lumen of the catheter; and
a therapeutic agent reservoir comprising at least one therapeutic agent and in fluid communication with the lumen of the drive shaft.

5. A method for local delivery of at least one therapeutic agent to a biological conduit, comprising:
providing a high-speed rotational drive shaft comprising a lumen therethrough and an eccentric abrading head thereon;
releasing the at least one therapeutic agent into the biological conduit near the eccentric abrading head;
influencing the at least one therapeutic agent radially outward toward the wall of the biological conduit; and
impacting the at least one therapeutic agent into the wall of the biological conduit.

6. The method of claim 5, further comprising:
initiating the high-speed rotational drive shaft comprising the eccentric abrading head thereon to high speed orbital rotation;
creating centrifugal forces radiating radially outward toward the wall of the biological conduit; and
using the centrifugal forces to influence the released at least one therapeutic agent radially outward toward the wall of the biological conduit; and
impacting the at least one therapeutic agent into the wall of the biological conduit.

7. The method of claim 5, further comprising:
initiating the high-speed rotational drive shaft comprising the eccentric abrading head thereon to high speed orbital rotation;
creating centrifugal forces radiating radially outward toward the wall of the biological conduit; and
impacting the released at least one therapeutic agent with the eccentric abrading head to influence the at least one therapeutic agent radially outward toward the wall of the biological conduit; and
impacting the at least one therapeutic agent into the wall of the biological conduit.

8. The method of claim 7, further comprising:
creating centrifugal forces radiating radially outward toward the wall of the biological conduit; and
using the centrifugal forces to influence the released at least one therapeutic agent radially outward toward the wall of the biological conduit; and
impacting the at least one therapeutic agent into the wall of the biological conduit.

9. The method of claim 8, further comprising:
providing a therapeutic delivery sheath comprising a lumen therethrough;
providing a therapeutic agent reservoir in fluid communication with the lumen of the therapeutic agent delivery sheath;
providing a pump in operative communication with the therapeutic agent reservoir; and
initiating the pump to pump the at least one therapeutic agent through the lumen of the therapeutic agent delivery sheath;
releasing the at least one therapeutic agent into the biological conduit, before initiating and/or during high-speed rotation of the drive shaft comprising the eccentric abrading head.

10. The method of claim 8, further comprising:
providing at least one aperture through the drive shaft, the at least one aperture in fluid communication with the lumen through the drive shaft;
providing a therapeutic delivery sheath comprising a lumen therethrough;
providing a therapeutic agent reservoir in fluid communication with the lumen of the drive shaft;
providing a pump in operative communication with the therapeutic agent reservoir; and
initiating the pump to pump the at least one therapeutic agent through the lumen of the drive shaft and radially outward through the at least one aperture;
releasing the at least one therapeutic agent into the biological conduit, before initiating and/or during high-speed rotation of the drive shaft comprising the eccentric abrading head.

11. The method of claim 5, wherein the at least one therapeutic agent is released into the biological conduit at a point proximal to the eccentric abrading head.

12. The method of claim 5, wherein the at least one therapeutic agent is released into the biological conduit at a point distal to the eccentric abrading head.

13. The method of claim 9, wherein the at least one therapeutic agent is released into the biological conduit at a point proximal to the eccentric abrading head.

14. The method of claim 9, wherein the at least one therapeutic agent is released into the biological conduit at a point distal to the eccentric abrading head.

15. The method of claim 10, wherein the at least one aperture is disposed proximal the eccentric abrading head.

16. The method of claim 10, wherein the at least one aperture is disposed distal to the eccentric abrading head.

17. The method of claim 16, further comprising the at least one aperture disposed proximal to the eccentric abrading head.

18. The method of claim 8, further comprising providing a therapeutic delivery sheath comprising a lumen therethrough;
providing a therapeutic agent reservoir in fluid communication with the lumen of the therapeutic agent delivery sheath;
initiating flow of the at least one therapeutic agent from the therapeutic agent reservoir through the lumen of the therapeutic agent delivery sheath;
releasing the at least one therapeutic agent from the lumen of the therapeutic agent delivery sheath into the biological conduit, before initiating and/or during high-speed rotation of the drive shaft comprising the eccentric abrading head.

19. A high-speed rotational atherectomy device for local delivery of at least one therapeutic agent to a biological conduit, comprising:
a guide wire having a maximum diameter less than the diameter of the artery;
a flexible elongated, rotatable drive shaft advanceable over the guide wire, the drive shaft having a rotational axis, the drive shaft rotatable at high rotational speeds;
an eccentric abrading head attached to the drive shaft, wherein the abrading head defines a drive shaft lumen therethrough and a hollow cavity, the drive shaft at least partially traversing the drive shaft lumen and wherein the at least one eccentric abrading head has a center of mass which is spaced radially away from the rotational axis of the drive shaft;
a flexible elongated catheter comprising a lumen,
a therapeutic agent delivery sheath comprising a lumen, the lumen comprising a distal end, the drive shaft slidably and rotatably disposed within the lumen of the therapeutic agent delivery sheath;
a therapeutic agent delivery lumen defined by the space between the catheter and the therapeutic agent delivery sheath; and
a therapeutic agent reservoir comprising the at least one therapeutic agent and in fluid communication with the therapeutic agent delivery lumen.
